(19) United States
(12) Patent Application Publication
(10) Pub. No.: US 2003/0208237 A1
(43) Pub. Date: Nov. 6, 2003

(54) Single-action method of activating and exposing user interface of medical device

(75) Inventors: Kelly J. Locke, Woodinville, WA (US); Shawn R. Bertagnole, Lake Stevens, WA (US); Richard J. Cardin, Duvall, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Medtronic Physio-Control Manufacturing Corp.

(21) Appl. No.: 10/139,942

(22) Filed: May 6, 2002

(51) Int. Cl. A61N 1/39

(52) U.S. Cl. 607/5

(57) ABSTRACT

The invention provides a method for sequentially activating a medical device and exposing at least a portion of the user interface of the medical device to an operator, in a single action to be performed by the operator. The medical device, for example, an automated external defibrillator (AED), includes a housing having a user interface and a lid that is coupled to the housing. The lid, when closed, is covering at least a portion of the user interface. In one embodiment, the medical device further includes an on/off button. The button is configured such that, when an operator depresses the button, it causes a switch to close to thereby activate the medical device, and further causes the lid to open via a latch mechanism.

### FIELD OF THE INVENTION

The present invention is related to a method of activating a medical device and, more particularly, to a method of activating a medical device and also exposing the user interface of the medical device to an operator in a single action to be performed by the operator.

### BACKGROUND OF THE INVENTION

The current trend in the medical industry is to make life-saving portable medical devices, such as automated external defibrillators (AEDs), more widely accessible. As the availability of portable medical devices continues to increase, more places will have these devices for use in emergency situations, such as in homes, police cars, worksites, and public gathering places. This increase also comes with the heightened likelihood that these portable medical devices will be used by people without medical training or people who are minimally trained in the handling of the medical devices. At the same time, the benefit of having life-saving medical devices immediately available in many places is not fully realized unless the medical devices can be promptly activated and used quickly in case of emergency. Therefore, a portable medical device, such as an AED, must be configured such that even a lay person can intuitively and quickly activate the medical device.

Once activated, a medical device may automatically instruct an operator how to properly operate the medical device via various user interface tools. For example, a medical device may include a voice command system, a screen command system, and/or various graphics visible to the operator. Additionally, as part of user interface tools, an AED typically stores therein a pair of defibrillation electrodes to be applied by an operator on the patient's body. Ideally, various user interface tools should be immediately available to the operator upon activation of a medical device so that the operator can, promptly after the activation of the medical device, access or follow instructions offered by the user interface tools to operate the medical device to save the patient's life. At the same time, at least some of the user interface tools should not be available until after the medical device is activated, to ensure that an instructional command, for example a voice prompt, of the medical device can timely guide the operator how to properly handle all the user interface tools. In the case of an AED, for example, a pair of electrodes should be immediately available to an operator upon activation of the AED but not prior thereto, because the operator may not know how to properly apply the electrodes on the patient's body. By having the AED activated first, the operator can follow the voice commands issued by the AED to apply the electrodes to the patient. This feature of activating a medical device first is particularly important when the medical device is likely to be used by a lay person who is not very familiar with the medical device and thus needs to rely on commands issued by the medical device to properly handle the medical device.

One prior attempt to address these needs is described in U.S. Pat. Nos. 5,797,969 and 6,083,246, which both describe an AED including a lid and a lid switch for detecting the open or closed position of the lid. In operation, when an operator opens the lid of an AED thereby exposing some of the user interface tools covered beneath the lid, the lid switch detects that the lid is opened and automatically activates the AED. This lid-activation method is based on the assumption that any operator will intuitively know to open the lid of an AED to activate the AED. In this regard, U.S. Pat. No. 6,083,246 specifically notes that its lid-activation method is preferable over the use of a depressible “power on” button to activate an AED because, when using a lid-activated AED, an operator does not have to “fumble with a ‘power on’ switch that may be confusingly placed among a multitude of switches.” However, in actuality, the lack of a “power on” button confuses many lay person operators who intuitively look for a “power on” button when faced with an unfamiliar medical device. In other words, the notion that opening a lid of a medical device automatically activates the medical device is not as intuitive as suggested by these patents.

### SUMMARY OF THE INVENTION

The present invention offers a method and system for sequentially activating a medical device and exposing at least a portion of the user interface of the medical device to an operator, in a single action to be intuitively performed by the operator.

In accordance with one aspect of the present invention, a medical device includes a housing having a user interface and a lid that is coupled to the housing. The lid, when closed, covers at least a portion of the user interface. For example, in the case of a semiautomatic AED, the user interface covered beneath the lid may include a pair of electrodes and a shock key which, when depressed by an operator, causes the AED to deliver a defibrillation shock. As another example, in the case of a fully automatic AED, the user interface may include a pair of electrodes but no shock button. The medical device further includes a mechanism for sequentially activating the medical device and opening the lid to expose the user interface covered by the lid. The mechanism is actuated in a single action to be performed by an operator.

For example, the mechanism may be formed of a button coupled to the housing, and the operator may activate the medical device and also open the lid by simply depressing the button a single time. Alternatively, the single action may be sliding a button, touching a button, or even having the operator's fingertip come close to a button. Further alternatively, the single action may be voicing an audible command into a speaker of the medical device.

In accordance with one specific aspect of the present invention, the mechanism for sequentially activating the medical device and opening the lid of the medical device includes generally two components: a medical device activation system for activating the medical device, and a lid-opening system for opening the lid to thereby expose the user interface covered by the lid. Both the medical device activation system and the lid-opening system are actuated by a single action to be performed by an operator. In one example, the medical device activation system includes a switch operably coupled to the underside of a depressible button. Further, the lid-opening system includes a latch that mechanically holds a spring-loaded lid in a closed position. In this configuration, when an operator depresses the button, a switch is closed to thereby activate the medical device. Additionally, depressing the button mechanically disengages the latch from the lid, to thereby cause the spring-loaded lid to pop open. In a further example, the lid-opening system may include other automatic lid-opening components, such as an electric motor or a gas-assist shock (strut) mechanism for opening the lid.

In still another aspect of the present invention, a fully automatic external defibrillator is provided, including a housing having a user interface and a lid that is coupled to the housing to cover at least a portion of the user interface. The fully automatic external defibrillator includes a switch for activating the defibrillator, but no button, switch, or other user-initiated mechanism for instructing the defibrillator to deliver a defibrillation shock. The switch is configured and arranged such that it is actuated to thereby activate the defibrillator when the lid is opened. The defibrillator further includes a suitable indicia or labeling visible to an operator when the lid is closed, to prompt the operator to open the lid to thereby activate the defibrillator. In this embodiment also, the operator's single action of opening the lid not only activates the filly automatic external defibrillator, but also exposes the user interface covered under the lid.

### BRIEF DESCRIPTION OF THE DRAWINGS

The foregoing aspects and many of the attendant advantages of this invention will become more readily appreciated by reference to the following detailed description, when taken in conjunction with the accompanying drawings, wherein:

FIG. 1 is a perspective view of a medical device comprising a defibrillator, which incorporates a mechanism for activating the medical device and exposing a user interface of the medical device in a single action, formed in accordance with the present invention;

FIG. 2 illustrates the medical device of FIG. 1, wherein a lid of the medical device is opened, exposing the user interface previously hidden beneath the lid;

FIG. 3 is a block diagram of several of the key components of the medical device of FIG. 1 comprising a defibrillator;

FIG. 4A is a schematic partial cross-sectional view of the medical device of FIG. 1, wherein some components are disproportionately enlarged for illustrative purposes;

FIG. 4B is a schematic partial cross-sectional view of the medical device of FIG. 1, wherein a lid of the medical device is being opened;

FIG. 5 illustrates one embodiment of a fully automatic external defibrillator, which incorporates a mechanism for activating the defibrillator and exposing a user interface thereof in a single action, formed in accordance with the present invention, wherein a lid is opened to expose the user interface covered beneath the lid;

FIG. 6 is a perspective view of another embodiment of a fully automatic external defibrillator, which incorporates a lid switch, formed in accordance with another embodiment of the present invention; and

FIG. 7 is the fully automatic external defibrillator of FIG. 6, wherein a lid is opened to expose a user interface previously covered beneath the lid.

### DETAILED DESCRIPTION OF THE PREFERRED EMBODIMENT

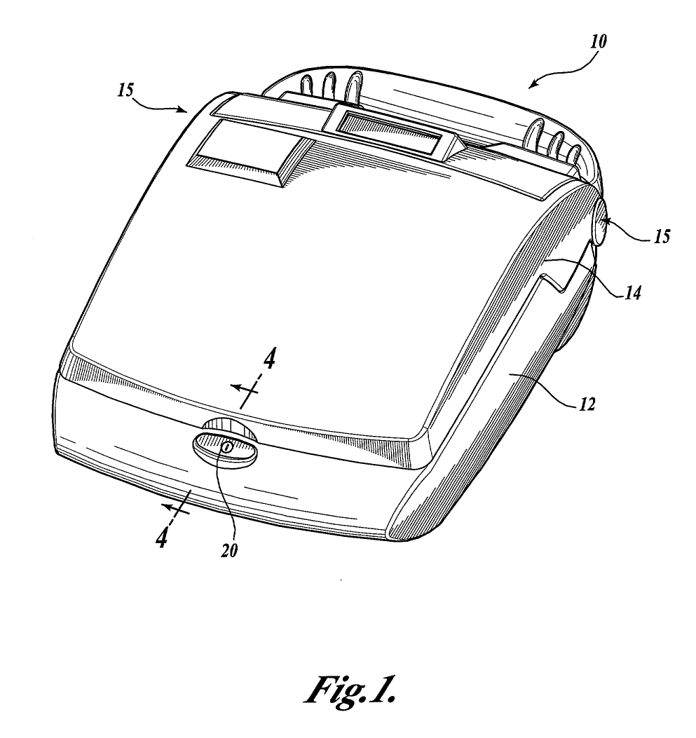

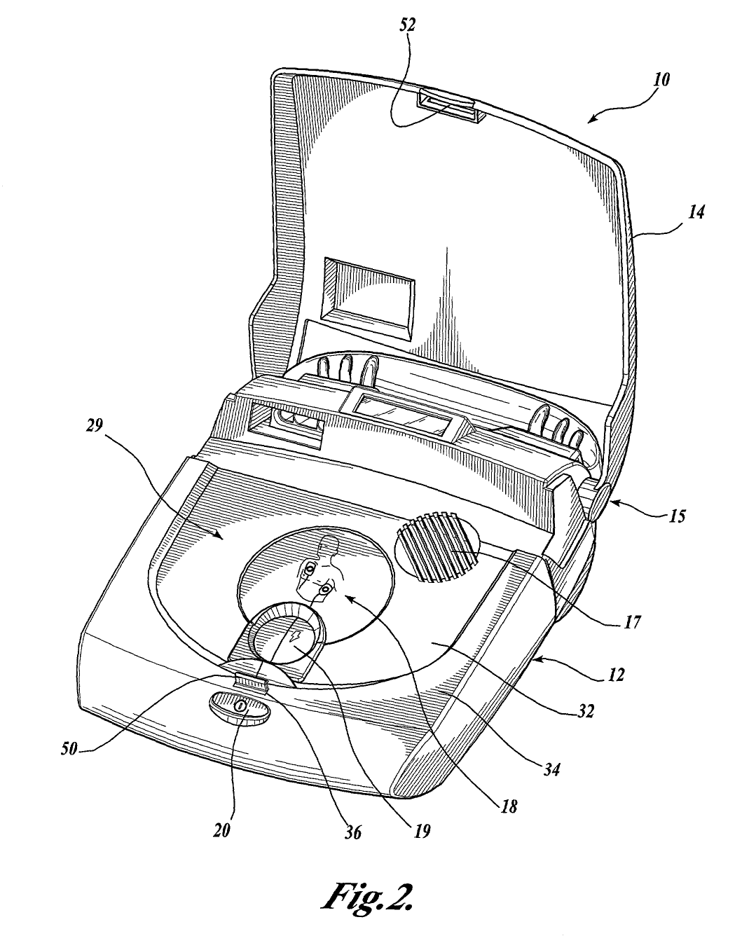

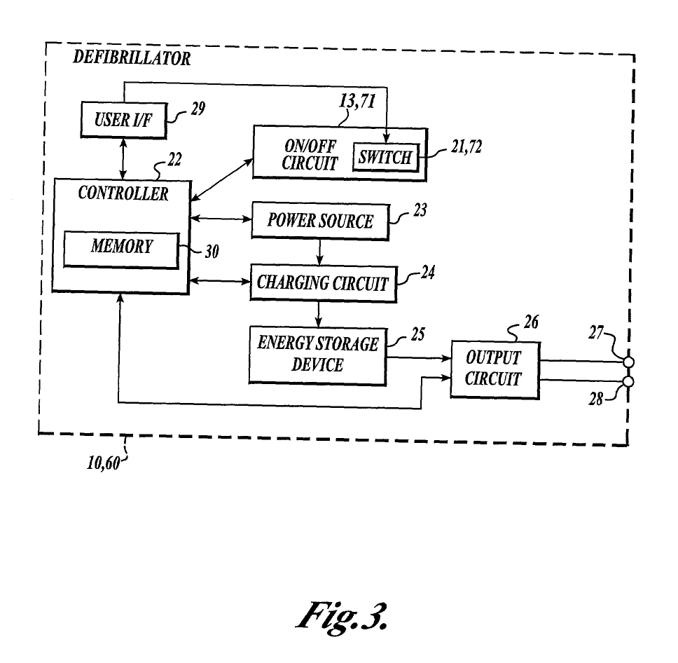

FIG. 1 illustrates a medical device embodied as a portable automated external defibrillator (AED) 10, suitable for incorporating the single-action activation/user interface exposure mechanism of the present invention. Although a defibrillator is used to describe this embodiment, in light of this disclosure, those of ordinary skill in the art will be able to implement the present invention with other types of medical equipment without undue experimentation. Also, the term “mechanism” as used in the present invention is not limited to a mechanical system, and may also include other systems based on electronics, magnetics, optics, etc. or any combinations thereof, as will be more fully appreciated by the following description. The AED 10 includes a housing 12 and a lid 14 coupled to the housing 12. In the illustrated embodiment, the lid 14 is pivotally coupled to the housing 12 at two pivot points 15 in a conventional manner, so that the lid 14 can be opened as shown in FIG. 2. The housing 12 contains electronics necessary for the operation of the AED 10, as will be described below in reference to FIG. 3.

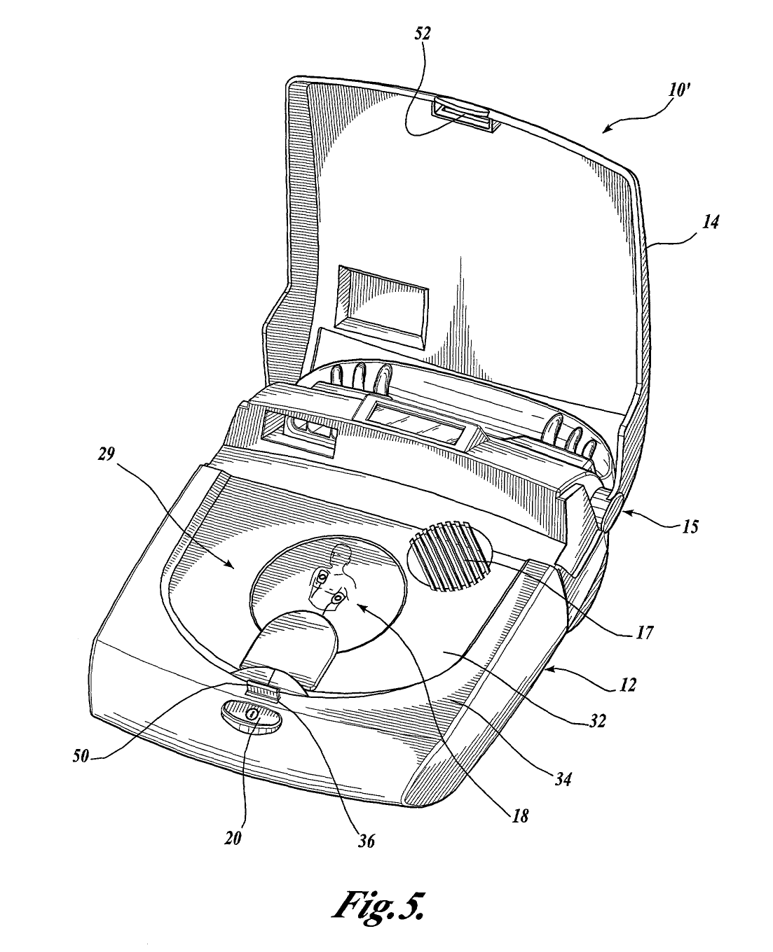

FIG. 2 illustrates the AED 10 of FIG. 1 when the lid 14 is opened. As illustrated, the housing 12 incorporates a user interface 29 including a speaker 17. In the present description, the term “user interface” is used to encompass any element that is used to send messages and/or instructions to and receives messages and/or instructions from an operator of the medical device 10, including any element that is to be physically handled by the operator. For example, the user interface 29 may include a pair of defibrillation electrodes (not shown) placed on the housing 12 to be handled by an operator. The user interface 29 may further include stationary or other graphics provided on the face of the housing 12, such as graphics 18 illustrating how to apply the defibrillation electrodes on a patient's body. Additionally, in the case of a semi-automatic AED, the user interface 29 of the AED 10 also includes a shock key 19, which is to be pressed by an operator to apply a defibrillation shock to the patient, as will be more fully described below. Alternatively, and as depicted in FIG. 5, in the case of a fully automatic AED, the shock key 19 may be eliminated so that the AED delivers a defibrillation shock to the patient automatically upon detection of a shockable heart rhythm as opposed to waiting for user initiation of a shock key or other trigger mechanism.

Turning now to FIG. 3, several of the key components of the AED 10 are described. It will be appreciated by those of ordinary skill in the art that the AED 10 may contain more components than those shown in FIG. 3. However, it is not necessary that all of these generally conventional components be shown in order to disclose an illustrative embodiment for practicing the present invention. FIG. 3 is a block diagram of an AED implemented as a defibrillator. The AED 10 includes an on/off circuit 13 including a switch 21, a controller 22, a power source 23, a charging circuit 24, an energy storage device 25, an output circuit 26, output electrodes 27 and 28, and a user interface 29.

The switch 21 is any type of a switch, for example an electric switch which, when closed (i.e., actuated), completes the built-in on/off circuit 13. As will be more fully described below, the switch 21 is closed by a user interface tool, for example, a depressible on/off button (20 in FIG. 1), which can be intuitively actuated by an operator. The on/off circuit 13 is coupled to the controller 22 and is configured, under the control of the controller 22, to activate/deactivate the medical device 10 when it is completed by the actuation of the switch 21. In the present description, the phrase “activating” a medical device means powering on the medical device, while “deactivating” a medical device means powering off the medical device. Various types of switches can be used for powering on and off a medical device, as will be apparent to those of ordinary skill in the art and also will be described later. With any type of a switch used, the switch is adapted to be actuated by a user interface tool (e.g., an on/off button). The controller 22 includes a microprocessor (not shown) such as, for example, a model 68332 available from Motorola, along with a memory 30. Preferably, the memory 30 includes random-access memory such as DRAM (dynamic random access memory) or SRAM (static random access memory), and nonvolatile memory such as a flash memory. The memory 30 is used to store software programs executed by the microprocessor (not shown) that control the operation of the AED 10.

The power source 23 is implemented with an internal battery. The internal battery can be recharged with a charging pack, such as a LIFEPAK® CR Series CHARGE-PAK™ available from Medtronic Physio-Control Corp. of Redmond, Wash. The charging circuit 24 is coupled to the power source 23. The energy storage device 25 is coupled to the charging circuit 24 and is implemented with a capacitor with a capacitance of about 190-200 μF. The output circuit 26 is coupled to the energy storage device 25 and is implemented in an H-bridge configuration, which facilitates generating biphasic defibrillation pulses. In operation, as well known in the art, under the control of the controller 22, the charging circuit 24 transfers energy from the power source 23 to the energy storage device 25, and the output circuit 26 transfers energy from the energy storage device 25 to the electrodes 27 and 28. The user interface 29 is implemented with conventional input/output devices, including, for example, the speaker 17 and shock key 19, as illustrated in FIG. 2. The speaker 17 outputs various commands and parameters necessary for the operation of the AED 10. The shock key 19 is to be depressed by the operator to trigger application of a defibrillation shock to the patient.

There are various types of defibrillators. For example, once defibrillation electrodes are connected to a patient, a fully automatic AED monitors and analyzes electrocardiogram (ECG) of the patient and, based on the ECG analysis, automatically delivers a defibrillation shock to the patient through electrodes without user intervention. Other AEDs, on the other hand, are semiautomatic in the sense that once the ECG analysis indicates that defibrillation is recommended, an operator is prompted to manually trigger delivery of a defibrillation shock to the patient, for example by pressing the shock key 19 as illustrated in FIG. 2. Therefore, the term defibrillator as used in the present description is intended to encompass various types of defibrillators.

As described above, when the lid 14 of the medical device 10 is closed, the lid 14 covers at least a portion of the user interface 29 included in the housing 12 (for example, the speaker 17, graphics 18, and shock key 19 in the case of the semi-automatic AED 10 illustrated in FIG. 2.) Also, in the case of a defibrillator, a pair of defibrillation electrodes (not shown) is typically stored underneath the lid 14. The present invention offers a single-action method and apparatus for sequentially activating the medical device (AED) 10 and opening the lid 14 to expose the user interface 29 covered by the lid 14. In other words, once the operator performs a single action to activate the medical device, the user interface of the medical device is immediately revealed to the operator.

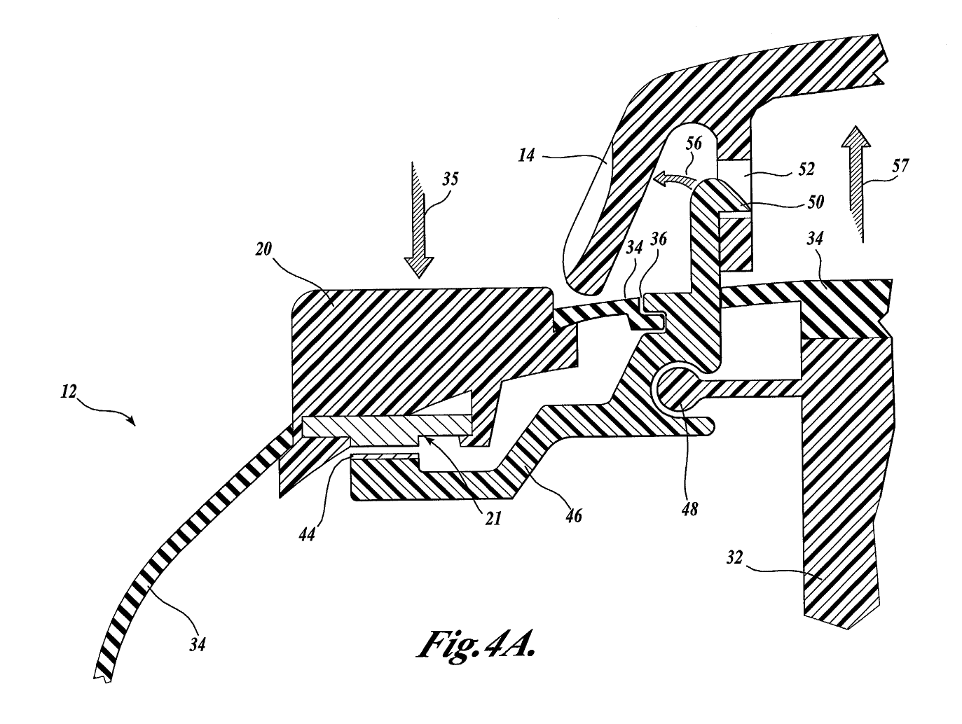

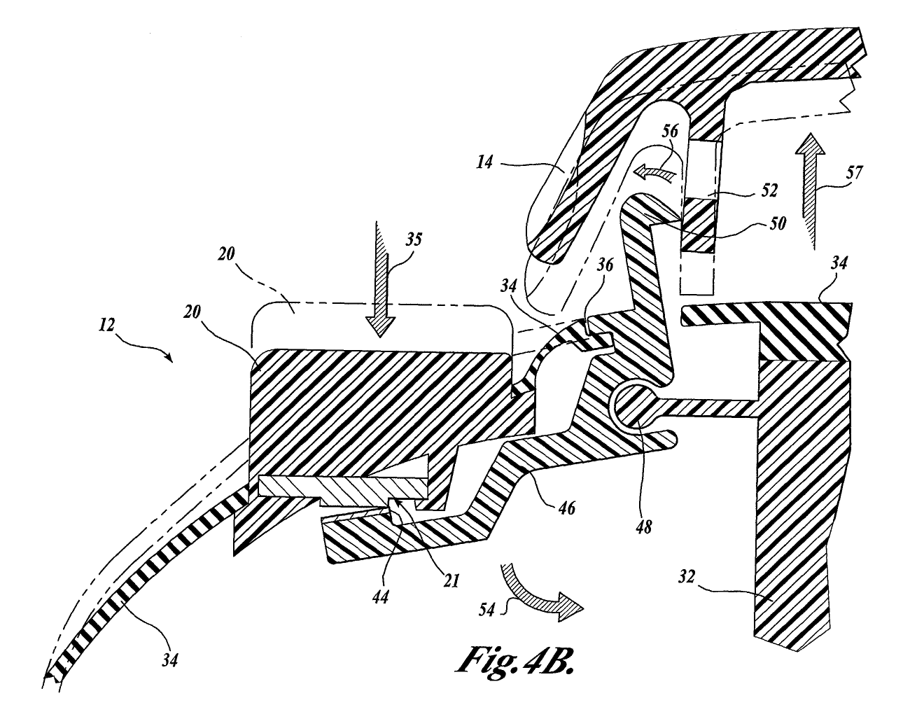

Returning to FIG. 1, in one embodiment of the present invention, the AED housing 12 includes an on/off actuator 20 which is sized and colored to be conspicuous to an operator. Though the on/off actuator 20 is illustrated to be in the form a button, the actuator may take various other shapes and forms, for example, a membrane, plate, bar, etc., as long as it is adapted to receive an operator's instruction to activate the AED 10. In the illustrated embodiment, the button 20 is configured so that depressing the button 20 sequentially activates the AED 10 and opens the lid 14 to expose the user interface 29 covered by the lid 14. Referring additionally to FIGS. 2 and 4A, the button 20 may be integrally formed with the housing 12. To that end, the housing 12 may be advantageously formed of a rigid component 32 made of, for example, plastic, and a resilient overlay component 34 made of, for example, urethane. In construction, the rigid component 32 is molded first, then the button 20 (also made of a rigid material such as plastic) is placed in a predetermined spaced-apart relation with respect to the rigid component 32. Thereafter, the resilient material from which the overlay component 34 is made is formed around the button 20 to integrally and firmly attach the button 20 to the rigid component 32. Thus constructed, the button 20 peripherally supported by the resilient overlay component 34 can be depressed relative to the rigid component 32, in a direction indicated by an arrow 35 (FIG. 4A). In other words, the resilient overlay component 34 provides a structural memory for the location of the button 20 as well as a spring that permits generally vertical movement of the button 20 relative to the rigid component 32 when the button 20 is depressed. Furthermore, use of the resilient overlay component 34 has additional advantages, such as protecting the medical device 10 from electrical or mechanical shocks and moisture.

Referring specifically to FIGS. 3 and 4A, an electrical switch 21 forming part of the built-in on/off circuit 13 is provided on the underside of the button 20. Specifically, the switch 21 in this embodiment is formed of a dome switch. The dome switch is “collapsed” when it is pressed against a contact member 44 (formed, for example, of rubber) as the button 20 is depressed in the direction of the arrow 35, thereby closing the switch 21. In short, the switch 21 is actuated by the button 20. As described above, the switch 21 is part of the built-in on/off circuit 13 coupled to the controller 22 of the medical device 10. When the switch 21 is closed, the on/off circuit 13 is completed, which in turn powers on the medical device 10 under the control of the controller 22. In one embodiment of the present invention, the switch 21 may be a toggle switch. In this embodiment, depressing the button 20 for the first time activates the medical device 10, and then depressing the button 20 for the second time deactivates the medical device 10.

Depressing the button 20 not only activates the medical device 10 but also opens the lid 14 to thereby expose the user interface 29 of the medical device 10 covered beneath the lid 14. As shown specifically in FIGS. 4A and 4B, the contact member 44 of the switch 21 is supported by one end of a latch 46, which in turn is pivotally supported at a hinge point 48. (The contact member 44 may be integrally formed with the latch 46, also.) The hinge point 48 is integrally formed from the rigid component 32 of the housing 12. The latch 46 defines a latch head 50 on an opposite end of the latch 46 from the switch 21. The latch head 50 is configured to pass through an aperture 36 defined in the resilient component 34 of the housing 12 so as to extend above the housing 12. The latch head 50 is further configured to engage with a hole 52 provided through the lid 14 in order to keep the lid 14 in a closed position. More specifically, in the illustrated embodiment, the lid 14 is spring loaded to be in an open position as shown in FIG. 2 by incorporating a pair of springs (not shown) in the pivot points 15 of the lid 14. Thus, in order to hold the lid 14 in a closed position as shown in FIGS. 1 and 4A, the springs are overcome by the latch head 50 engaging with the hole 52 of the lid 14. Additionally, when closing the lid 14, the aperture 36 defined through the resilient overlay component 34 provides a structural memory for the position of the latch 46 in the latched position (i.e., the lid-closed position.)

In operation, referring specifically to FIG. 4B, when the button 20 is depressed, the switch 21 is closed to thereby activate the medical device 10, as described above. Furthermore, continuing depressing the button 20 will cause the latch 46 to pivot around the hinge point 48 of the housing 12 in the direction of an arrow 54. At the same time, the latch head 50 will pivot in the direction of an arrow 56 and disengage from the hole 52 of the lid 14, thereby releasing the spring-loaded lid 14 to an open position. Accordingly, the single action by an operator of depressing the button 20 will sequentially activate the medical device 10 and open the lid 14 to expose the user interface 29 of the medical device 10 to the operator. Once the medical device 10 is activated, as well known in the art, suitable software of the medical device controls the operation of the device, including offering commands, such as voice commands, to guide the operator through the proper operation of the device. The voice commands will be issued through the speaker 17, which was previously hidden beneath the lid 14 in the illustrated embodiment.

One advantage of the construction described above is that even if the lid 14 is opened or removed altogether from the housing 12, the medical device 10 will not be activated as long as the button 20 is not pressed, and the lid 14 may be readily reinstalled by an operator. In other words, the button 20 controls the activation (and deactivation) of the medical device 10, regardless of the position, or even the presence, of the lid 14.

As will be clearly understood from the foregoing disclosure, a single-action method and apparatus for activating a medical device and also exposing the user interface of the medical device can be readily incorporated in various types of medical devices other than defibrillators, e.g., electrocardiogram monitoring devices, drug infusion devices, etc. Further, as will be also appreciated by those skilled in the art, numerous alternatives to the button actuation system described above are possible. For example, the button 20 may be configured to slide in a generally horizontal direction to close a switch and thus activate and subsequently open the lid of the medical device. Though the button 20 is illustrated as integrally formed with the housing 12, it may be provided as a separate element that is depressibly coupled to the housing 12 (via a spring, for example) or slidably coupled to the housing 12 (via a sliding slot or rail, for example) using any suitable construction. Also, though the latch 46 is illustrated as a separate element from the housing 12, it may be integrally formed with the housing 12. Further alternatively, the latch 46 may consist of a plurality of subcomponents that are linked and supported by a plurality of pivot (hinge) points, respectively. As an example, one subcomponent may pivot about another subcomponent that pivots about a hinge point. Of course, when a plurality of subcomponents are used, the pivoting movement of all subcomponents are preferably coordinated so that a single-action actuation of the button 20 will cause the switch 21 to close and subsequently open the lid 14. Though the hinge point 48 is illustrated to be integrally formed with the housing 12, it may be provided as a separate element coupled to the housing 12 also.

Still further, the switch 21 need not be located on the button 20 as illustrated in FIG. 4A, but may be located at any location on the latch 46 or any subcomponents of the latch, the housing 12, or on any element used in constructing the mechanism for sequentially activating the medical device 10 and exposing the user interface of the medical device upon a single action performed by the operator. Also, the method for spring-loading the lid 14 is not limited to what is disclosed herein, and, for example, springs may be arranged in various ways. Further alternatively, the lid 14 need not be spring-loaded to be opened upon a single action to be performed by the operator. For example, the lid 14 may be configured to be opened by a leveraged mechanical system, motor, a gas-assist shock (strut) mechanism, or any other suitable mechanism as long as it functions to open the lid 14 in response to a single action performed by the operator.

As briefly described above, the single-action method and apparatus of activating a medical device and also exposing the user interface of the medical device may be based on various types of switches, such as electrical switches, mechanical switches, optical switches, magnetic switches, or switches based on a thermal or infrared (IR) sensor, proximity sensor, capacitive sensor, motion sensor, or audio sensor, or some combination thereof. For example, when an operator touches or when the operator's finger comes close to a thermal sensor, the heat of the operator's finger is detected by the sensor to thereby trigger activation of the medical device. The proximity sensor switch is actuated (closed) when the electric field that it produces is disturbed by a human fingertip contacting or coming close to the proximity sensor. The capacitive sensor switch is actuated when the capacity of its capacitor is changed (e.g., grounded) by a human fingertip contacting the capacitor. The motion sensor switch, often based on optics, is actuated when an optical beam it produces is disturbed by a human fingertip (or perhaps other trigger) contacting or coming close to the motion sensor. An audio sensor switch may be configured to activate the medical device when a certain command above a certain volume is voiced into a microphone (not shown) attached to the medical device. Other types of switches may also be used, such as magnetic switches including a reed relay switch and a Hall-effect sensor switch. In summary, any switch that may be actuated in a single action (e.g., depressing, sliding, or touching a button, plate, or membrane, or voicing a command into a microphone) to be performed by an operator can be used. When the medical device is activated via a non-mechanical switch, for example via an IR sensor or an audio sensor, the controller 22 (FIG. 3) is configured to cause the lid 14 to open via some automatic mechanism, for example, by an electric motor (not shown) coupled to the lid 14.

As briefly described above, when a toggle-type switch is used, the medical device 10 may be deactivated by depressing the on/off button 20 one more time after the medical device is activated. Alternatively or additionally, the medical device 10 may be deactivated by operation of software. For example, it may be “timed out” based on a predetermined time lapse from a triggering event. As one specific example, the medical device 10 in the form of an AED may be configured to be deactivated when the controller 22 (FIG. 3) determines that a pair of electrodes are not properly placed on the patient for more than 15 minutes (based on the measurement of unusually high impedance across the electrodes).

The single-action or “single-button” method and apparatus of the present invention for activating and exposing the user interface of the medical device may be applied to both semi-automatic and fully-automatic defibrillators. FIG. 5 illustrates a fully automatic external defibrillator 10′ incorporating the on/off button 20. The configuration of the fully automatic external defibrillator 10′ is substantially equivalent to the configuration of the semi-automatic external defibrillator 10 illustrated and described in reference to FIGS. 1 through 4B above, except that the fully automatic external defibrillator 10′ does not include the shock key 19. As briefly described above, the fully automatic external defibrillator 10′ is configured to monitor the patient's ECG through a pair of defibrillation electrodes, analyze the ECG, and then, based on the ECG analysis, automatically deliver a defibrillation shock to the patient through the electrodes without user intervention. Therefore, by definition, a fully automatic external defibrillator does not include any user interface element to be actuated by an operator to deliver a defibrillation shock, such as the shock key 19 included in the semi-automatic external defibrillator 10 of FIG. 2. In other respects, the fully automatic external defibrillator 10′ is generally equivalent to the semi-automatic external defibrillator described above, and thus the block diagram of FIG. 3 can be referred to as illustrating the overall electronics of the fully automatic external defibrillator 10′ also.

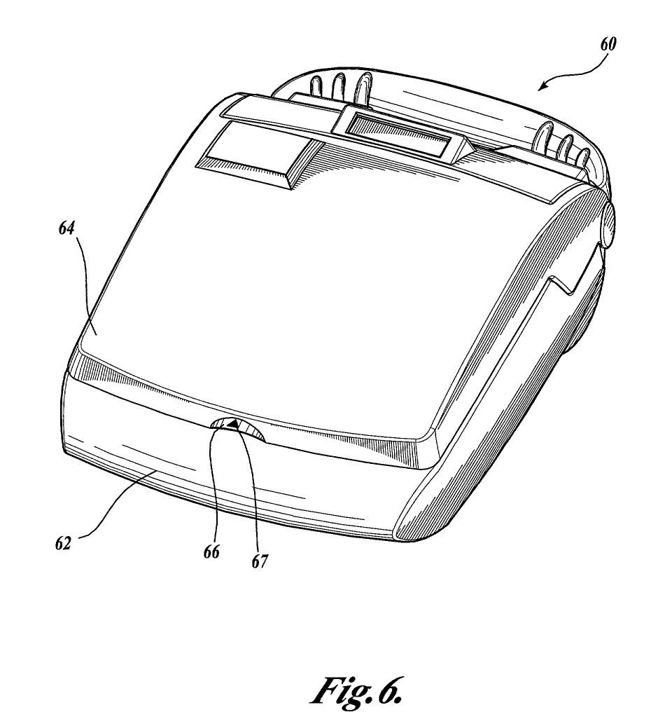

Referring next to FIG. 6, another embodiment of a fully automatic external defibrillator formed in accordance with the present invention is illustrated, wherein the on/off button 20 is eliminated and the fully automatic external defibrillator is activated and the user interface exposed by the single action of opening the lid. While it is well known to apply a lid-activation mechanism in semi-automatic defibrillators, it is not well known to do so in fully automatic defibrillators. In fact, conventional wisdom indicates that a fully-automatic defibrillator without an on/off button may prove to be oversimplified for the untrained user who is expecting at least some level of interaction with the device. However, as described in more detail below, with appropriate labeling for the operator, such shortcomings in a lid-activated (as opposed to on/off button activated), fully automatic defibrillator can be overcome.

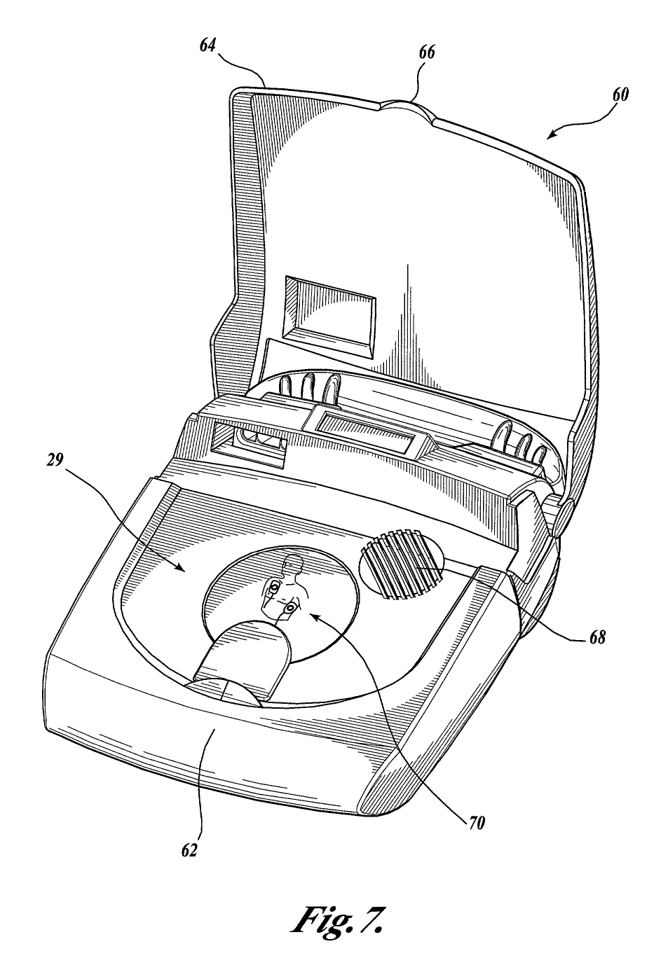

Specifically, in FIGS. 6 and 7, a medical device 60 in the form of a fully automatic external defibrillator is provided. As before, the configuration of the fully automatic external defibrillator 60 is equivalent to the configuration of the semiautomatic external defibrillator described in reference to FIGS. 1-4B above, except that the fully automatic external defibrillator 60 does not include the shock key 19 or on/off button 20 included in the semi-automatic external defibrillator shown in these FIGURES. As described above, the fully automatic external defibrillator 60 is configured to automatically deliver a defibrillation shock to the patient without user intervention and thus, by definition, does not include any user interface element for delivering a defibrillation shock, such as the shock key 19 in FIG. 2. In addition, in the embodiment illustrated in FIGS. 6 and 7, the fully automatic external defibrillator 60 also does not include an on/off or power-on button, such as the button 20 shown in FIG. 1, for activating the device. In other respects, the fully automatic external defibrillator 60 is generally equivalent to the semi-automatic external defibrillator described above, and thus the block diagram of FIG. 3 can be referred to as illustrating the overall electronics of the fully automatic external defibrillator 60 also. The configuration and operation of a fully automatic external defibrillator are well known in the art and are thus not described in detail in the present description, except for the single-action “lid-activation” method as applied in a fully automatic external defibrillator in accordance with the present invention.

In FIG. 6, the fully automatic external defibrillator 60 includes a housing 62 and a lid 64 coupled to the housing 62. The fully automatic external defibrillator 60 also includes a switch 21 (FIG. 3) for activating the defibrillator, which is actuated when the lid 64 is closed, as will be more fully described below. The lid 64 includes a lift handle 66 that is configured to be lifted by an operator to thereby open the lid 64. Additionally, the fully automatic external defibrillator 60 includes an indicia 67 visible to an operator when the lid is closed, for instructing or prompting the operator to lift the lid 64. In the illustrated embodiment, the indicia 67 comprises an “up” triangle, graphically instructing an operator to lift up the handle 66. The indicia 67, however, is not limited to this particular embodiment, and may include other graphical indicia (an “up” arrow, a red dot, etc.) or text indicia (“LIFT HERE,” “PULL UP,” etc.) designed to reasonably instruct an even untrained operator to lift or open the lid 64. With the use of such explicit indicia, the operator can be guided to promptly lift the lid 64 to thereby activate the medical device.

FIG. 7 illustrates the fully automatic external defibrillator 60 of FIG. 6 when the lid 64 is opened. As before, opening the lid 64 will expose various user interface 29 components that were previously covered beneath the lid 64, for example, the speaker 68 and the graphics 70. As before, once activated, the fully automatic external defibrillator 60 starts issuing a voice prompt via the speaker 68, and in particular, prior to and during application of a defibrillation shock, instructs the operator to stand clear of the patient, as well known in the art.

Referring additionally to FIG. 3, the fully automatic external defibrillator 60 includes an on/off circuit 71 including a sensor switch 72 that is configured and arranged to sense the position of the lid 64 as opened or closed. The switch 72 is actuated when the lid is opened or closed, to thereby complete the on/off circuit 71, which in turn, under the control of the controller 22, activates or deactivates the fully automatic external defibrillator 60 based on the detected position of the lid 64. For example, in one embodiment, the on/off circuit 71 is configured to activate the fully automatic external defibrillator 60 when the switch 72 detects the lid 64 is opened, and to deactivate the defibrillator 60 when the switch 72 detects the lid 64 is closed. Sample lid switches suitable for use in the present embodiment include a relay switch such as a magnetic reed relay switch, a Hall-effect sensor switch, and any other electro/mechanical-type, magnet/mechanical-type, semiconductor-type, or optical-type switches known in the art.

Thus, when an operator, being prompted by the indicia 67, opens the lid 64 of the fully automatic external defibrillator 60, the lid switch 72 detects opening of the lid 64 and completes the on/off circuit 71, which in turn activates the defibrillator 60. At the same time, the act of opening the lid 64 necessarily exposes any user interface tools 29 that are covered underneath the lid 64. Therefore, in this embodiment also, a single action to be performed by the operator (i.e., opening the lid 64) both activates the medical device and also exposes the user interface of the medical device to the operator. As described above, the fully automatic external defibrillator 60 may be further configured to be deactivated upon closing the lid 64. Accordingly, when an operator closes the lid 64, the lid switch 72 detects closing of the lid 64 and completes the on/off circuit, which in turn deactivates the defibrillator 60. However, in other embodiments, the defibrillator 60 may be deactivated by operation of software, e.g., on a “time-out” basis.

While the preferred embodiments of the invention have been illustrated and described, it will be appreciated that various changes can be made therein without departing from the spirit and scope of the invention.

The embodiments of the invention in which an exclusive property or privilege is claimed are defined as follows:

1. A medical device comprising:
a housing having a user interface;
a lid coupled to the housing to cover at least a portion of the user interface; and
a mechanism for sequentially activating the medical device and opening the lid to expose the user interface covered by the lid, the mechanism comprising a switch for activating the medical device, and the mechanism being actuated in a single action.

2. The medical device of claim 1, wherein the mechanism comprises a button coupled to the housing.

3. The medical device of claim 2, wherein the single action comprises depressing the button.

4. The medical device of claim 3, wherein the housing of the medical device comprises:
a first component portion formed of a rigid material; and
a second component portion formed of a resilient material, wherein the button is depressibly supported by the second component portion.

5. The medical device of claim 2, wherein the single action comprises sliding the button.

6. The medical device of claim 2, wherein the single action comprises touching the button.

7. The medical device of claim 2, wherein the switch for activating the medical device is coupled to the button and configured to be closed to activate the medical device when the button is actuated in a single action, the mechanism further comprising a latch for opening the lid, the latch being coupled to the button and configured to cause the lid to open when the button is actuated in the same single action that closes the switch.

8. The medical device of claim 7, wherein the lid is spring loaded.

9. The medical device of claim 7, wherein the latch is pivotally supported at a hinge point formed from the housing, the latch comprising a first end configured to engage with the lid when the lid is closed and a second end configured to come in contact with the button when the button is actuated in the same single action that closes the switch.

10. The medical device of claim 7, wherein the housing of the medical device comprises:
a first component portion formed of a rigid material; and
a second component portion formed of a resilient material, wherein the button is depressibly supported by the second component portion and the second component portion defines an aperture through which the latch extends.

11. The medical device of claim 1, wherein the mechanism comprises an audio sensor, and the single action comprises voicing a command to be detected by the audio sensor.

12. The medical device of claim 1, wherein the mechanism comprises an infrared (IR) sensor.

13. The medical device of claim 1, wherein the mechanism comprises a proximity sensor, and the single action comprises a human fingertip contacting or coming close to the proximity sensor to disturb an electric field produced by the proximity sensor.

14. The medical device of claim 1, comprising a defibrillator.

15. The medical device of claim 14, comprising a semi-automatic external defibrillator.

16. The medical device of claim 14, comprising a fully automatic external defibrillator.

17. The medical device of claim 1, wherein the switch comprises an electrical switch.

18. The medical device of claim 1, wherein the switch comprises a mechanical switch.

19. The medical device of claim 1, wherein the switch comprises an optical switch.

20. The medical device of claim 1, wherein the switch comprises a magnetic switch.

21. A single-action method of activating a medical device, comprising:
(a) providing a medical device comprising:
(i) a housing having a user interface, and
(ii) a lid coupled to the housing to cover at least a portion of the user interface; and
(b) sequentially activating the medical device and opening the lid to expose the user interface covered by the lid in a single action performed by an operator.

22. The method of claim 21, wherein the single action comprises depressing an element coupled to the medical device.

23. The method of claim 21, wherein the single action comprises sliding an element coupled to the medical device.

24. The method of claim 21, wherein the single action comprises touching or coming close to an element coupled to the medical device.

25. The method of claim 21, wherein the single action comprises voicing an oral command to an audio sensor coupled to the medical device.

26. A medical device comprising:
a housing having a user interface;
a lid coupled to the housing to cover at least a portion of the user interface;
a medical device activation system for activating the medical device, the medical device activation system comprising a switch that is configured to be closed by a single action to be performed by an operator; and
a lid-opening system for opening the lid to thereby expose the user interface covered by the lid, the lid-opening system being configured to be actuated subsequent to the switch being closed by the same single action performed by the operator that closes the switch.

27. The medical device of claim 26, wherein the medical device activation system further comprises an actuator for closing the switch to activate the medical device, the switch being coupled to the actuator and configured to be closed when the actuator is actuated in a single action; and
the lid-opening system comprises a latch for opening the lid, the latch being coupled to the actuator and configured to cause the lid to open when the actuator is actuated in the same single action that closes the switch.

28. The medical device of claim 27, wherein the actuator comprises a button.

29. The medical device of claim 27, wherein the lid is spring loaded.

30. The medical device of claim 27, wherein the lid is motor driven.

31. A fully automatic external defibrillator, comprising:
a housing having a user interface;
a lid coupled to the housing to cover at least a portion of the user interface; and
a switch for activating the defibrillator, the switch being actuated upon opening the lid.

32. The defibrillator of claim 31, further comprising an indicia instructing an operator to open the lid, the indicia being visible to the operator when the lid is closed.

33. A fully automatic external defibrillator, comprising:
a housing having a user interface;
a lid coupled to the housing to cover at least a portion of the user interface;
a switch for activating the defibrillator, the switch being actuated upon opening the lid; and
an indicia instructing an operator to open the lid, the indicia being visible to the operator when the lid is closed.

34. The defibrillator of claim 33, wherein the indicia comprises a graphic label.

35. The defibrillator of claim 33, wherein the indicia comprises a text label.

36. The defibrillator of claim 33, wherein the switch is a Hall effect switch.

37. The defibrillator of claim 33, wherein the switch is a relay switch.

38. The defibrillator of claim 37, wherein the switch is a magnetic reed relay switch.

39. the defibrillator of claim 33, wherein the switch is a semiconductor switch.

40. The defibrillator of claim 33, wherein the switch is an optical switch.

41. The defibrillator of claim 33, wherein the switch is a mechanical switch.
